(19) United States
(12) Patent Application Publication
(10) Pub. No.: US 2010/0317053 A1
(43) Pub. Date: Dec. 16, 2010

(54) PROCESS MACHINERY FOR FEEDING PRE-TREATED LIGNOCELLULOSIC MATERIALS INTO BIOREACTORS FOR BIO-FUELS AND BIOCHEMICALS

(75) Inventors: Bertil STROMBERG, Diamond Point, NY (US); Thomas PSCHORN, Sherbrooke (CA); Peter MRAZ, Klosterneuburg (AT); Serge GENDREAU, Saint-Lazare (CA)

Correspondence Address: NIXON & VANDERHYE, PC, 901 NORTH GLEBE ROAD, 11TH FLOOR, ARLINGTON, VA, 22203, US

(73) Assignee: ANDRITZ INC., Glens Falls, NY (US)

(21) Appl. No.: 12/795,869

(22) Filed: Jun. 8, 2010

Related U.S. Application Data
(60) Provisional application No. 61/186,947, filed on Jun. 15, 2009.

(51) Int. Cl. C12P 1/00

(52) U.S. Cl. 435/41

(57) ABSTRACT

Methods for mixing a pretreated cellulosic biomass feedstock using a centrifugal mixer prior to reactions in a bioreactor.

### RELATED APPLICATION

This application claims priority to U.S. Provisional application Ser. No. 61/186,947, filed on Jun. 15, 2009, the entirety of which is incorporated by reference

### BACKGROUND OF THE INVENTION

The present invention relates to pre-treatment of cellulosic biomass feedstocks, such as, agricultural residues (which may include corn stalks, corn stover, hulls, cereal straws, etc.); energy plants (such as high yielding grasses like Switchgrass, Miscanthus, EnergyCane, etc.); and/or forest or sawmill residues (such as wood chips, shredded thinnings, etc.) for the further production of bio-fuels and chemicals.

Several different pre-treatment system options may be conventionally used to modify the structure of cellulosic feed material to extract the carbon sugars for the further production of bio-fuels. These pre-treatment systems include, for example, acid dilute hydrolysis, steam explosion, and concentrated acid hydrolysis. Depending on the enzyme, organism or chemical (such as an acid or a base) being used in the pre-treatment system, saccharification and fermentation may be separate process steps or simultaneous process steps (SSF-concepts). For example, a pre-treated feedstock may be fed to special designed reactors (e.g., a CSR or constant stir reactor or any other type of liquefaction reactor).

In conventional paper-making, it is known to use a fluffer to add additives to a fiber stock suspension. See, e.g., U.S. Pat. Nos. 7,169,258; 6,077,396; and 5,630,909, all of which are incorporated by reference herein.

### BRIEF DESCRIPTION OF THE INVENTION

In aspect, the invention relates to a method a preparing a biomass feedstock for subsequent reaction and conversion into a biofuel.

In another aspect, an embodiment may relate to a method for feeding biomass to a reactor for conversion into biofuel. The method includes two steps: feeding a pretreated cellulosic biomass feedstock comprising precursors and reactants for bioreactions via a compression device to a centrifugal mixer comprising a rotor and a stator; and rotating the rotor to disintegrate larger particle agglomerates of the pretreated cellulosic biomass feedstock.

### BRIEF DESCRIPTION OF THE DRAWINGS

FIG. 1 is an illustration of an embodiment.

FIG. 2 is another illustration of an embodiment.

### DETAILED DESCRIPTION OF THE INVENTION

Because bio-reactor(s) may be operated under a very low positive pressure to significantly reduce the entrainment of undesired organisms, it may be necessary to feed pretreated material into the reactor using a special device. For example, compression devices like screw presses, plug feeders or similar devices often in combination with sealed conveyors with screw augers may feed reactants to the top of a reactor (e.g., a down-flow reactor).

Pretreated feedstock may contain a larger amount of precursors and reactants for bioreactions (e.g., sugars). In many instances, these sugars (or other released substances during the pretreatment) pack and/or compress the feedstock into larger particles that stick or floc together (e.g., through the before mentioned sugars) after being compressed (or just by being conveyed) in screw-auger-type conveyors or similar devices.

In an aspect, an embodiment generally relates to an apparatus for feeding reactants to a reactor. This apparatus may operate in conjunction with a device that may cool and/or mix the pretreated feedstock with water, enzymes, organisms (such as, but not limited to, bacteria), chemicals and/or a mixture thereof.

In an aspect, an embodiment may generally relate to a special disc-type fluffer, which may be similar to a typical chemical mixer in a mechanical pulp bleaching system or as being presently used, e.g., for mechanical pulping or for medium density fiberboard (MDF) refining applications. In other aspects, an embodiment may generally relate to a medium consistency mixer/discharger, possibly operating in reverse to take advantage of the centrifugal effect. This device of these embodiments could be fed and discharged under atmospheric conditions. Alternatively (and perhaps preferably), they may be fed and then also discharged under slight pressurized conditions, so as to facilitate disintegrating larger particle agglomerates and dispersing the pretreated feed-stock in relative fine and small particles while at the same time possibly introducing and mixing water, enzymes, organisms, chemicals and/or a mixture thereof.

It may be possible to convey conditioned feedstock from the mixing device via a conveying device into a reactor, and it may also be possible that this mixing device is installed directly above a reactor, preferably a down-flow reactor.

In an aspect, an embodiment of the apparatus may contain a rotating disc, e.g., a horizontal disc with a vertical driven shaft or a vertical rotating disc with a horizontal shaft. The disc may have replaceable plate segments with special teeth, such as, for example, small squares, pyramids, and/or vanes on the rotating disc. These teeth may disintegrate larger particle agglomerates (e.g., fluff), mix-in a suspension (e.g., a suspension sprayed into the apparatus), etc. The apparatus may also propel the material towards the discharge by centrifugal force.

In an embodiment, the mixing apparatus may also be equipped with an injection port for optional addition of a liquid and/or gaseous stream. The added liquids may, for example, facilitate or accelerate downstream digestion into simple sugars and/or downstream fermentation. The liquid stream may consist of water, catalyzing agents, acidic agents, including, but not limited to, sulfuric acid, enzymes, and/or other agents formulated to enhance pre-digestion of cellulosic feed material or to enhance conversion into biofuel. Alternatively or conjunctively, gas(ses) may be added to the mixing apparatus. The gas(ses) may facilitate adjusting the temperature (e.g., cooling) and/or particle separation in addition to possibly maintaining a proper (e.g., elevated) pressure in the mixer and/or downstream reactor.

Pretreated feedstock or similar material from a feeding device (which could also be a blow-cyclone with or without a discharge device, such as a scraper, for example) may be discharged into a conveyor (e.g., a screw conveyor or the like). From there, it may then be dropped into (e.g., via gravity and/or other means) the mixing apparatus feed screw (which may be a full flight screw or ribbon feeder, separately driven or directly bolted to the rotating disc). The feed screw may convey the material towards the center of the rotating disc, and the teeth-type pattern on the plate or vanes (e.g., similar to a pump or a fan) may propel the material towards the periphery of the machine (e.g., through centrifugal force).

These plates with teeth or vanes may either be cast via conventional casting techniques, welded from disparate parts, or even possibly machined into shape. One or more segments may comprise a plate. For instance, partial (e.g., half) or full discs may be mounted to the rotating disc, i.e., rotor, and the stationary disc, i.e., stator.

It may also be possible that vanes are part of the rotating disc. On the opposite site (e.g., the stationary part of the housing) there may be also vanes installed to further enhance the propelling effect.

It may also be possible that a (smaller) ring of refining plates may be installed at the periphery. This set of refining plates may be cast, welded, or machined. Partial or full discs may be mounted to the rotating disc (e.g., similar to a refiner, but only on the outer radius).

The housing (e.g., the area between feed screw and discharge) may be rated for a pressure similar than the one inside the downstream bio-reactor (e.g., approximately 0.2 to 1 bar). There may be a seal (e.g., lip seal, mechanical seal, special stuffing box, etc.) mounted between the shaft and the mixer housing.

From the mixing apparatus pretreated material, e.g. lignocellulosic material, may be then fed to the following process stage. This further stage may include the bio-reactor, such as, for example, a reactor designed to facilitate an enzymatic hydrolysis or microorganism treatment and/or fermentation to produce the desired sugars and ultimately alcohols, such as, for example, ethanol, and/or other kinds of biochemicals or similar applications.

In an aspect, the apparatus described herein may be an existing machine being used in a new way. For example, there is an existing type of machine that is used in mechanical pulping systems to disintegrate larger particle agglomerates and as a mixer in bleaching area of the mechanical pulping system, always entering the machine at atmospheric conditions. As described herein, however, the machine may be used directly after the pressurized equipment and would be used to disintegrate larger particle agglomerates of, e.g., at least partially hydrolyzed material.

In an aspect, at least certain embodiments involving the use of the apparatus as an enzyme mixer.

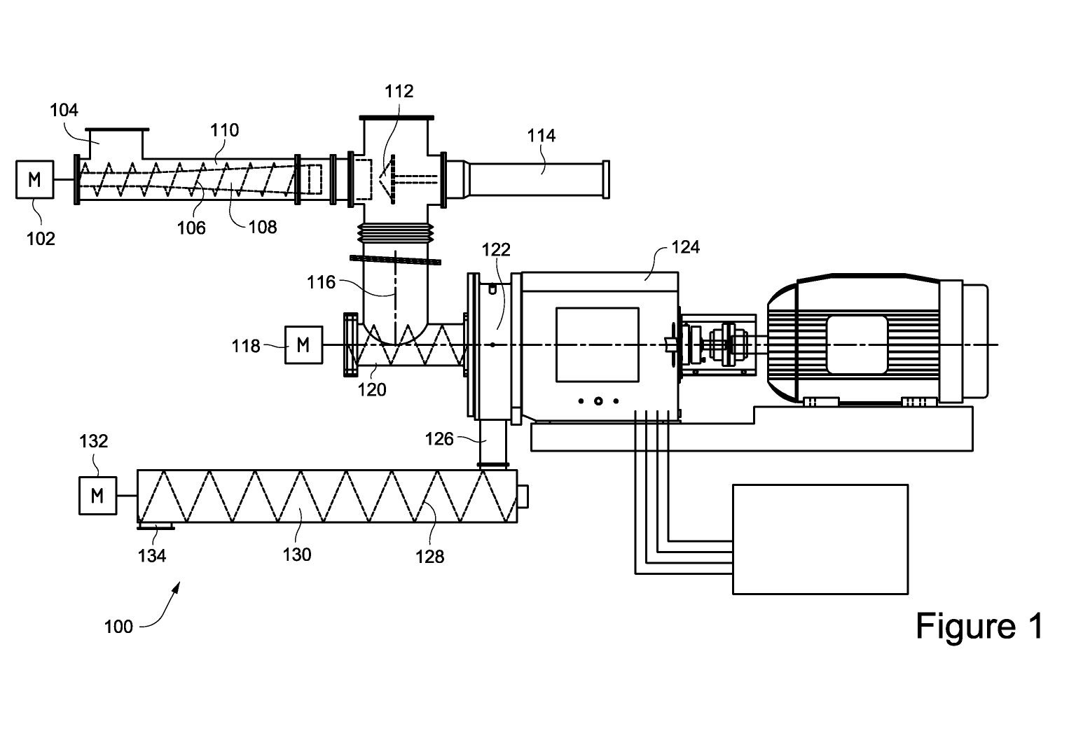

FIG. 1 illustrates an embodiment of an apparatus in accordance with an aspect of the present invention. Mixing system 100 has an inlet 104 for receiving lignocellulosic material for conversion to biofuel. Plug screw feeder 108 contains a compaction screw with auger-like blades 106. Plug screw feeder 108 is rotated by motor 102, such that the lignocellulosic material moves along conduit 110 away from inlet 104 and down through conduit 116. Plunger 112 and blow back valve 114 may prevent blow back up through conduit 116. From conduit 116, the material travels through conduit 120 via a ribbon feeder or plug screw feeder rotated by motor 118. This permits the lignocellulosic material to be fed to mixer 124, which contains a chamber 122 housing a rotor and stator (not shown) for mixing and/or disintegrating larger particle agglomerates within the lignocellulosic material. Liquid may be added to chamber 122, and the liquid stream may consist of water, catalyzing agents, acidic agents, including, but not limited to, sulfuric acid, enzymes, organisms, and/or other agents formulated to enhance pre-digestion of cellulosic feed material or to enhance conversion into biofuel. Alternatively or conjunctively, gas(ses) may be added to chamber 122. The gas(ses) may facilitate adjusting the temperature (e.g., cooling) and/or particle separation in addition to possibly maintaining a proper (e.g., elevated) pressure in the mixer 124 and/or downstream reactor.

The material then exits mixer 124 via conduit 126, which connects to conduit 130, in which the material moves via a conveyor, ribbon feeder, and/or screw feeder 128 powered by motor 132. The material exits via outlet 134 for further transport to a reactor (not shown).

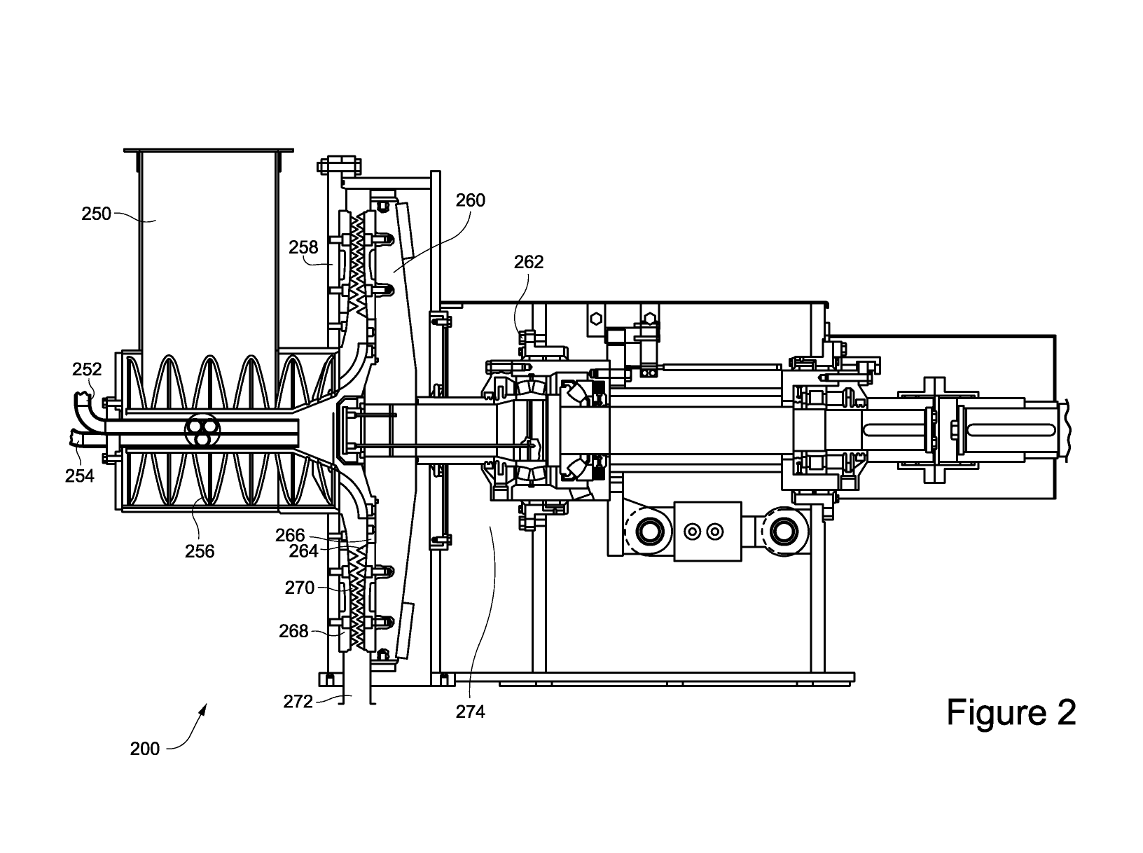

FIG. 2 illustrates an embodiment of an apparatus in accordance with an aspect of the present invention. Mixing system 200 has an inlet 250 for receiving lignocellulosic material for conversion to biofuel. Screw feeder 256 pushes the material into mixer 274. Mixer 274 has a stator 258 (which is substantially stationary during operation) and rotor 260 (which is connected to shaft 262 and rotates during operation). Plate 266 with teeth 264 is attached to rotor 260. A substantially mirrored (e.g., complementary) plate 268 with teeth 270 is attached to stator 258. The teeth may be configured in any suitable configuration, so long as at least some mixing occurs during operation and so long as the teeth on the stator do not contact the teeth on the rotor during operation.

Liquids (including, for example, water, enzymes, organisms, chemicals and/or mixtures thereof) may be added to mixer 274 via conduit 252 and/or conduit 254, the precise placement of which may be altered in various embodiments. Alternatively or conjunctively, gas(ses) may be added to mixer 274 via conduit 252 and/or conduit 254. The gas(ses) may facilitate adjusting the temperature (e.g., cooling) and/or particle separation in addition to possibly maintaining a proper (e.g., elevated) pressure in the mixer 274 and/or downstream reactor. The material exits via outlet 272 for further transport to a reactor (not shown).

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method for feeding biomass to a reactor for conversion into biofuel, the method comprising the steps of:
feeding a pretreated cellulosic biomass feedstock comprising precursors and reactants for bioreactions via a compression device to a centrifugal mixer comprising a rotor and a stator, and
rotating the rotor to disintegrate larger particle agglomerates of the pretreated cellulosic biomass feedstock.

2. The method according to claim 1, further comprising the subsequent steps of feeding the pretreated cellulosic biomass feedstock to a reactor via a second compression device and then subjecting the pretreated cellulosic biomass feedstock to at least one of enzymatic hydrolysis, a treatment with one or more microorganisms, or fermentation.

3. The method according to claim 2, further comprising adding a liquid to the centrifugal mixer such that the liquid and the pretreated cellulosic biomass feedstock are mixed prior to feeding the pretreated cellulosic biomass feedstock to the reactor.

4. The method according to claim 3, wherein the liquid comprises at least one of water, enzymes, organisms, or chemicals.

5. The method according to claim 2, further comprising adding a gas to the centrifugal mixer such that the gas and the pretreated cellulosic biomass feedstock are mixed prior to feeding the pretreated cellulosic biomass feedstock to the reactor.

6. The method according to claim 5, further comprising adjusting a temperature of the pretreated cellulosic biomass feedstock with the gas.

7. The method according to claim 6, further comprising pressurizing the pretreated cellulosic biomass feedstock with the gas.

8. The method according to claim 1, wherein the precursors and reactants for bioreactions include sugars.

9. The method according to claim 1, further comprising an initial step of pretreating a cellulosic biomass feedstock with at least one of water, enzymes, organisms, or chemicals to form the pretreated cellulosic biomass feedstock.

10. The method according to claim 1, further comprising pressurizing the centrifugal mixer to atmospheric conditions.

11. The method according to claim 1, further comprising pressurizing the centrifugal mixer to a pressure between 0.2 and 1 bar.

12. The method according to claim 1, wherein the rotor comprises a full or partial disc having teeth for mixing that is mounted to a rotating disc, and wherein the stator comprises a full or partial disc having teeth for mixing that is mounted to a stationary disc.

13. The method according to claim 12, wherein the teeth attached to the rotor are complementary with the with teeth attached to the stator.

13. The method according to claim 1, wherein the rotor comprises vanes or blades to propel the pretreated the pretreated cellulosic biomass feedstock from a center of the centrifugal mixer to an outer edge of the centrifugal mixer.

14. The method according to claim 1, wherein the compression device comprises a screw press, plug feeder, or a sealed conveyor with a screw auger.

15. The method according to claim 1, wherein the compression device comprises a screw press, plug feeder, or a sealed conveyor with a screw auger.

16. The method according to claim 2, wherein the second compression device comprises a screw press, plug feeder, or a sealed conveyor with a screw auger.

17. The method according to claim 1, wherein the cellulosic biomass feedstock comprises at least one of agricultural residues, energy plants, or forest or sawmill residues.

18. The method according to claim 1, wherein the agricultural residues comprise corn stalks, corn stover, hulls, or cereal straws.

19. The method according to claim 1, wherein the energy plants comprise grasses including Switchgrass, Miscanthus, or EnergyCane.

20. The method according to claim 1, wherein the forest or sawmill residues comprise wood chips or shredded thinnings.
